(19) United States
(12) Patent Application Publication
(10) Pub. No.: US 2015/0257808 A1
(43) Pub. Date: Sep. 17, 2015

(54) METHOD AND APPARATUS FOR DETERMINING WHETHER TO DELIVER A SUBSTANCE TO BONE

(71) Applicant: Spinal Generations, LLC, Mokena, IL (US)

(72) Inventor: Patrick J. Sweeney, Flossmoor, IL (US)

(21) Appl. No.: 14/209,302

(22) Filed: Mar. 13, 2014

(51) Int. Cl. A61B 17/88

(52) U.S. Cl. CPC A61B 17/8805

(57) ABSTRACT

A method for determining an indication for use of a bone support substance in a bone includes providing a surgical device for providing rotational movement of a surgical tool, determining a torque measurement associated with the rotation of the surgical tool as it is moving through a portion of the bone, and using the torque measurement to determine whether the portion of the bone has a characteristic that indicates the need for a bone support substance. Determining a torque measurement may include determining a motor torque generated to cause rotation of the surgical tool as it is moving through the portion of the bone. Determining a torque measurement may also include determining a drilling torque, wherein the drilling torque is the force created by the portion of the bone that acts against the motor torque, as the surgical tool is moving through the portion of the bone.

### BACKGROUND

The present invention relates generally to the field of implanting devices in a bone of a patient, and more particularly to determining an indication for using a bone support substance to support the implantation of a device in a bone.

Various conditions or characteristics of a bone of a patient may limit proper and secure implantation of a bone fixation element or prosthetic device. In such situations, it is helpful to support the implantation and engagement with the surrounding bone by using a bone support substance such as a bone void filler, bone cement, or other medication. In many situations, however, a medical professional performing the implantation may not be apprised of the conditions or characteristics of that patient's particular bone structure, and therefore cannot know ahead of time whether a bone support substance would be useful. In these cases, it may not be indicated until after a first attempt at implantation has failed.

One way in which a medical professional may learn of the condition of the bone is prior to implantation, performing common radiographic or other bone assessment procedures to determine whether a condition, such as osteoporosis or osteomalacia, or other condition lowering the quality of the bone, is present. This requires a step separate from and prior to the actual implantation process. Typically, no bone assessment method is employed during an implantation or bone preparation, and within the normal course of the procedure, to determine the quality and characteristics of the bone that would therefore allow the surgeon to determine that a bone support substance would be necessary to support the implantation and fixation of the fixation device or prosthesis.

### SUMMARY

One embodiment of the invention relates to a method for determining an indication for use of a bone support substance in a bone. The method includes providing a surgical device configured for providing rotational movement of a surgical tool, determining a torque measurement associated with the rotation of the surgical tool as it is moving through a portion of the bone, and using the torque measurement to determine whether the portion of the bone has a characteristic that indicates the need for a bone support substance.

Another embodiment of the invention relates to a method for implanting a prosthetic component. The method includes the steps of providing a surgical tool configured for rotational movement; determining a torque measurement associated with the rotation of the surgical tool as it is moving through a portion of the bone; and using the torque measurement to determine whether the portion of the bone has a characteristic that indicates the need for a bone support substance to secure the prosthetic component in a proper position; providing the bone support substance the portion of the bone if indicated by the torque measurement; implanting the prosthetic component in the proper position in the portion of the bone.

Another embodiment of the invention relates to a method for determining an indication for use of a bone support substance in a bone. The method includes measuring a value related to the rotation of a surgical tool, determining whether the value is within a predetermined range of values, wherein a value outside the predetermined range indicates a characteristic of the bone; determining, based on the characteristic of the bone, whether a bone support substance should be introduced to the bone.

### BRIEF DESCRIPTION OF THE FIGURES

The disclosure will become more fully understood from the following detailed description, taken in conjunction with the accompanying figures, wherein like reference numerals refer to like elements.

FIG. 1A depicts a healthy proximal femur bone, for which methods according to an exemplary embodiment may be used.

FIG. 1B depicts an osteoporotic proximal femur bone, for which methods according to an exemplary embodiment may be used.

FIG. 2 depicts a bone nail positioned in a proximal femur and the bone being prepared to receive a secondary fixation device.

FIGS. 3A-3E depict exemplary surgical tools for use with the methods according to an exemplary embodiment.

FIG. 4 is a schematic depiction of the surgical device and surgical tool according to an exemplary embodiment.

FIG. 5 is a flowchart of a method for determining an indication for use of a bone support substance in a bone, according to an exemplary embodiment.

FIG. 6 is a flowchart of a method for implanting a prosthetic component, according to an exemplary embodiment.

FIG. 7 is a flowchart of a method for determining an indication for use of a bone support substance in a bone, according to a second exemplary embodiment.

### DETAILED DESCRIPTION

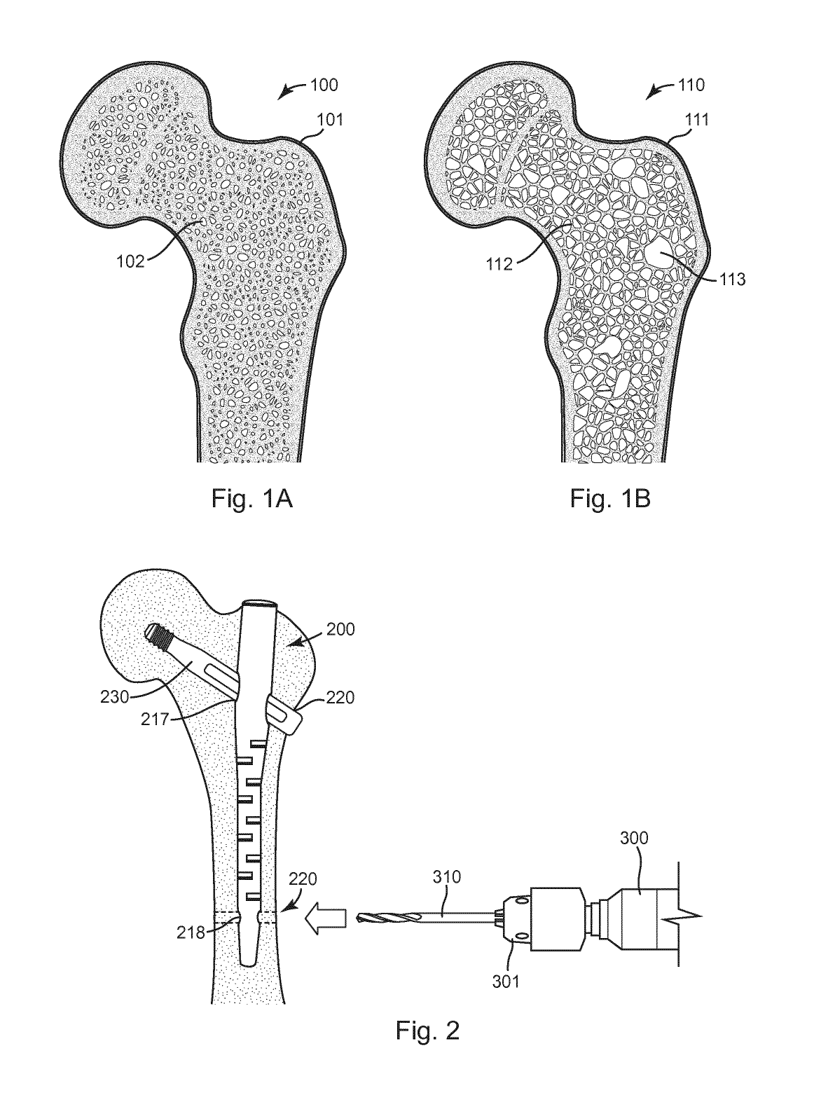

Referring to FIG. 1, depicted is a comparison between a healthy bone and an osteoporotic bone, in particular, a healthy proximal femur 100 and an osteoporotic proximal femur 110. Osteoporosis is a bone disease characterized by a decrease in the density and mass of the bone. Such a decrease can lead to an increased risk of fracture of the bone. Some risk factors for osteoporosis include aging, being female, low body weight, life stages (menopause), smoking, and some medications. Exercise, medications intended to improve or prevent osteoporosis, and diets with appropriate amounts of calcium and vitamin D can help to treat or prevent osteoporosis. The diagnosis of osteoporosis is typically made using conventional radiography techniques. In dual energy X-ray absorptiometry, which is a widely regarded method for diagnosing osteoporosis, a patient's bone mineral density is given a t-score. A bone receiving a t-score more than 2.5 standard deviations below that of a young, healthy adult woman (which serves as the healthy bone reference mean) is considered to have osteoporosis. A bone receiving a t-score between −2.5 and −1.0 standard deviations below the healthy bone reference mean is considered to have osteopenia, which may be considered a precursor to osteoporosis. As shown in FIG. 1B, an osteoporotic bone, such as proximal femur 110, has pores 113 that are larger than those of a healthy bone, such as proximal femur 100. This is one reason that osteoporotic bones are more susceptible to breakage than normal, healthy bone. Another similar condition affecting the strength of the bone is osteomalacia in which the bones are softer than normal.

The bone mineral density and/or the softness of one's bone not only affects the strength of the bone in normal conditions, but also influences the bone's ability to receive and maintain orthopedic fixation devices, such as bone screws, nails, tacks, pins, and the like, or other prosthetic devices, such as joint prostheses. Similarly, otherwise brittle, soft, weak, or damaged bones may also face problems for implanted devices in the bone. In these cases, certain substances and medications may be used to promote bone growth and support the contact between the bone and the implanted devices. Such substances may be a bone support substance, such as a bone void filler, a bone cement, or other bone medications intended to promote bone growth and fill in the gaps and spaces of a bone and/or between a bone and an implanted device. These substances may be used to more securely position and fix the device in the bone.

FIG. 2 depicts a bone nail 200 received in the proximal femur. The bone nail 200 is an exemplary embodiment of a bone nail or other bone fixation device that is configured to stabilize a bone or multiple bones by holding two or more bones or bone pieces in a fixed spatial relationship with respect to each other. The bone nail may be coupled to the exterior surface of the bone or may be disposed in an interior portion (e.g., the medullary canal) of the bone. The bone nail may be secured with one or more fixation devices, such as bone screws that engage both the bone nail and the surrounding bone tissue. Other exemplary embodiments of a bone fixation device are described in U.S. patent application Ser. No. 12/427,520, filed Apr. 21, 2009, titled “Method and Device for Delivering Medicine to Bone,” which is hereby incorporated by reference in its entirety.

As shown in FIG. 2, the bone nail 200 may be secured in the desired position in the bone by secondary fixation devices, such as bone screw 230, implanted into the bone and passing through a bore 218 in the bone nail 200. Often, a passage 220 is first formed in the bone to receive the bone screw 230, as shown. The passage may be created using a drill 300 having a drill bit 310.

Some bones are less conducive to securely retaining a bone fixation device or prosthetic devices, such as bone nail 200 and bone screw 230. One reason may be that the bone is affected by osteoporosis or osteomalacia as discussed above, or is otherwise too brittle, soft, weak, damaged, or does not have the density to support implanted devices. In these cases, it would be useful to have an indication of the bone's ability to securely retain the bone fixation devices or other inserts or prosthetic devices that may be implanted in the bone. Further, doing so while preparing the bone to receive the implanted devices or while doing the implantation itself, streamlines the process by combining the test/assessment of bone with the implantation. According to an exemplary embodiment, drill 300, or other surgical device holding a tool that contacts the bone, may be used to perform the assessment of the bone during bone preparation and/or implantation of these bone fixation devices and/or prosthetic devices, by determining certain values associated with the rotation of the surgical tool by the surgical device. In this way, the surgeon or other user receives an indication that the bone may require a bone support substance, in order to more successfully maintain the bone fixation device, prosthetic device, or other implanted device in a proper and desired position.

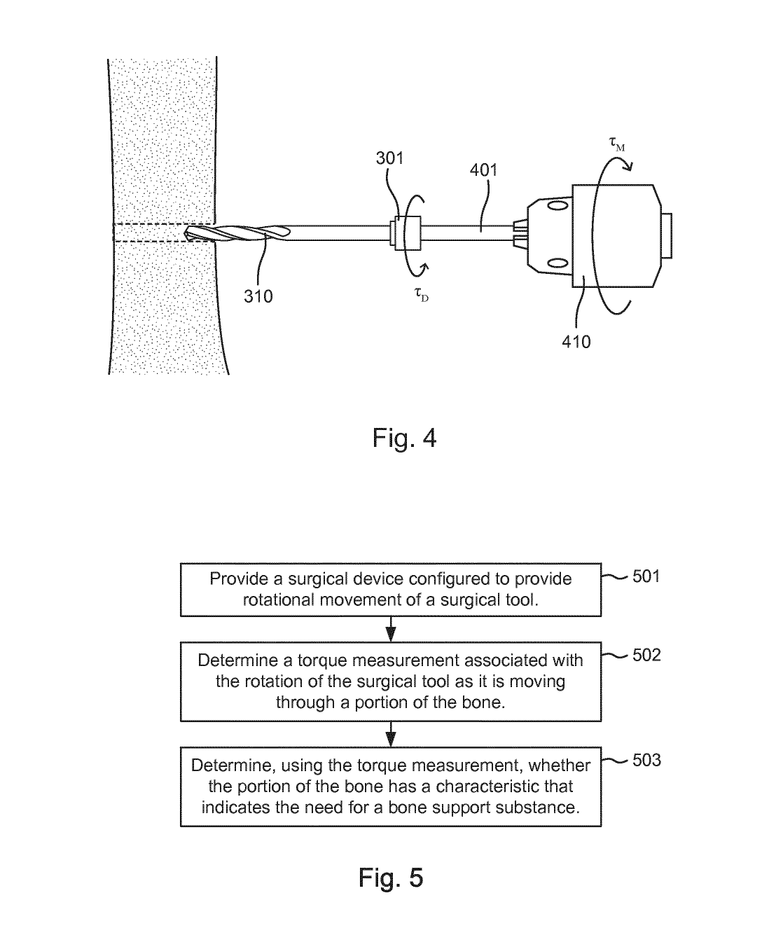

Still referring to FIG. 2, a drill 300 is used to move a drill bit through a portion of the bone. An exemplary drill will include, among other elements, a motor (410 in FIG. 4), a drive member (401 in FIG. 4), and a connecting mechanism including a chuck 301, which couples the surgical tool, such as drill bit 310 to the drive member. The drill 300 may be configured, as described below, to provide information and/or determine certain values that indicate a characteristic of the portion of the bone in order to make the assessment of the bone.

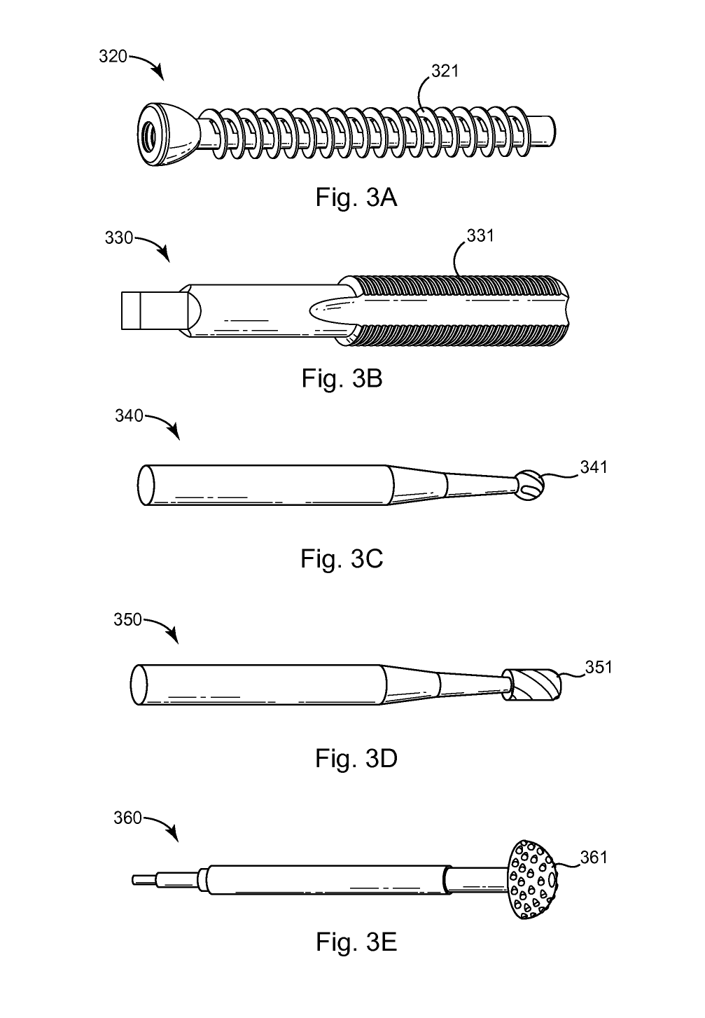

It should be understood that while reference herein is often made to a drill causing rotation of a drill bit to form a bore in a bone for receiving a fixation device, other rotating tools could be used in accordance with the methods described. The methods described are suitable for any surgical device that imparts rotational motion on a surgical tool in order to perform a desired activity. For example, the surgical tool attached to a surgical device similar to the drill 300 described above could be a screw 320, tapping tool 330, or device having a distal end configured for bone cutting or shaping, such a burrs 340, 350, or a reamer 360. In the case of a screw 320, shown in FIG. 3A, the surgical device is a screw driver device and instead of a surgical tool acting on the bone, the screw 320 itself acts on the bone when it is being implanted in the bone. In some situations, it may be useful to prepare the bone ahead of time for receiving the screw, such as through use of the drill/drill bit combination, or by using a tapping tool 330. As shown in FIG. 3B, tapping tool 330 includes male threads 331 which are used to form female thread recesses in the bone. The threads 321 of the screw 320 can more easily engage the bone as it is inserted into the bore created by the tapping tool 330. FIGS. 3C and 3D depict burring devices 340 and 350. A burr, such as burrs 340 and 350, can be used in bone sculpting, particularly in using the distal tip 341 and 351 for preparing a bone to receive a prosthetic implant, such as a joint prostheses. A reamer 360, such as that shown in FIG. 3E is also used to sculpt the bone in preparation to receive an implant component. A reamer 360 is generally intended to create a larger and deeper recess than that created using a burr. Similar to drill 330, a surgical device carrying these surgical tools, or others, may be used to cause rotational action of the tools which are manipulated about the bone to carry out the desired activity, and the present descriptions relate to any surgical devices and tools which can perform these activities.

According to various exemplary embodiments, measurements may be performed using the surgical device to make the assessment of the bone during bone preparation or device implantation. FIG. 4 is a schematic diagram of a surgical device drive shaft 401 connected to a surgical device motor 410. The drive shaft is coupled, such as through a mechanism including a chuck 301, to a surgical tool, such as drill bit 310. As shown, various torque forces are present as the surgical tool is rotated to perform the desired activity. The torque measurements are indicative of the forces necessary to cause rotation of the surgical tool in the target portion of the bone, and can therefore provide an assessment as to the quality of the bone. Assessing the quality of the bone allows the surgeon or other user to determine whether a bone void filler or bone cement, should be used.

Still referring to FIG. 4, a first torque is the motor torque (τ_{M}). The motor torque is the rotational force applied to the drive shaft 401 by the motor 410 of the surgical device. In one embodiment, the motor torque can be determined at any time during the activity of the surgical device. The motor torque can be determined by measuring the amount of current used to power the motor and calculate the torque therefrom. In this way, motor torque determinations may not require the use of external or separate sensors, and in particular, without the use of an external torque sensor. The measure of the motor torque considers the opposing torque forces generated by the bone as the tool is moving through the bone (described as drilling torque, below) and also any losses incurred by the system.

FIG. 4 also indicates a drilling torque (τ_{D}). The drilling torque is the torque generated by the bone that is applied to the surgical tool (in opposition to the motor torque) as it is moving and rotating within the bone. Though the term “drilling” is used to describe this force, it should be understood that this torque is applied to the surgical tool whether during drilling, burring, reaming, tapping, etc., and for any of a variety of surgical tools that rotate within the bone. The drilling torque can be determined using a torque sensor attached to the housing of the surgical device or built into the surgical device.

In other exemplary embodiments, a torque wrench may be used to monitor the application of torque when rotating a tool in the bone, or may be used to specify/limit the amount of torque provided to the tool, to determine the minimum value of torque required to rotate the tool.

In addition to torque measurements, other values may be useful to make the assessment of the bone. In another exemplary embodiment, a value related to the rotation of the surgical tool is the angular velocity of the surgical tool as it is moving through the bone.

The value determined during the activity of the surgical tool indicates a characteristic of the portion of the bone through which the tool is passing. For example, a lower than expected (e.g. relative to healthy bone) torque value would indicate that not much force is needed to rotate the surgical tool, and therefore, that the bone may be very soft, or brittle. In an angular velocity embodiment, a higher than normal angular velocity for a given motor output would indicate the ease at which the tool is working through the bone and thus, that the bone may be soft and in need of support. Furthermore, such indications would then suggest to the surgeon or medical professional that bone void filler or bone cement may be necessary to maintain the proper position of an implanted device.

The values determined during the procedure using the surgical tool may be made at particular points in the bone, or may be determined continuously throughout the procedure. For example, a surgeon may be most interested in determining the torque measurement only at the cortical bone 101 portion and the cancellous bone 102 portion, as identified in FIG. 1A. In other examples, a surgeon may wish to generate values associated with the rotation of the surgical tool at different depths into the bone when drilling a bore to receive a fixation device, or at different points in the surface when using a burr to prepare a bone to receive a prosthetic component.

According to other exemplary embodiments, the surgical device may be equipped with a display feature, wherein the values associated with the rotation of the surgical tool may be displayed continuously (e.g. instantaneous values), or the values may be displayed only at particular times or locations. The distinct times or locations may be pre-determined and pre-selected by the user, or may be pre-set by the device. The surgical device may also be equipped to provide an alert to the user to indicate, for example, that a pre-determined value of torque has been reached (e.g. that the torque value has dropped below a critical value, and bone void filler is now indicated). The alert may be an audible alert such as a beep or a ring, or may be a visual alert such as an LED indicator.

Using the exemplary methods herein, surgeons or other medical professionals may be able to use the determined values associated with the rotation of surgical tools, such as torque or angular velocity (e.g. using rotations per minute as an indicator), to create a systematic database for reference in making future assessments of bone quality for receiving implanted devices. After associating the values determined during the activity with the success or failure of bone fixation or prosthetic device, one can determine a critical point or a range of values based on the critical point. In subsequent procedures, if a determined value, such as torque or angular velocity, does not fit in the critical range, the surgeon can know without further assessment that a bone support substance is required for success of the implantation.

Referring to the flowchart of FIG. 5, described is a method for determining an indication for use of a bone support substance, such as a bone void filler in a bone in accordance with an exemplary embodiment. In step 501, a surgical device is provided. The surgical device is configured to provide rotational movement of a surgical tool. The surgical device may cause rotation of one of a variety of surgical tools, such as a burr, a screw, a drill, a reamer, or a tapper. In step 502, a torque measurement associated with the rotation of the surgical tool as it is moving through a portion of the bone is determined As described above, the torque measurement may be one of a motor torque or a drilling torque, or any rotational force associated with the rotation of the surgical tool. In step 503, the torque measurement is used to determine whether the portion of the bone has a characteristic that indicates the need for a bone support substance m such as a bone void filler. For example, if the torque value is low it may indicate the presence of soft, brittle, or damaged bone which may further indicate that a bone support substance may be needed to support the strength of the bone.

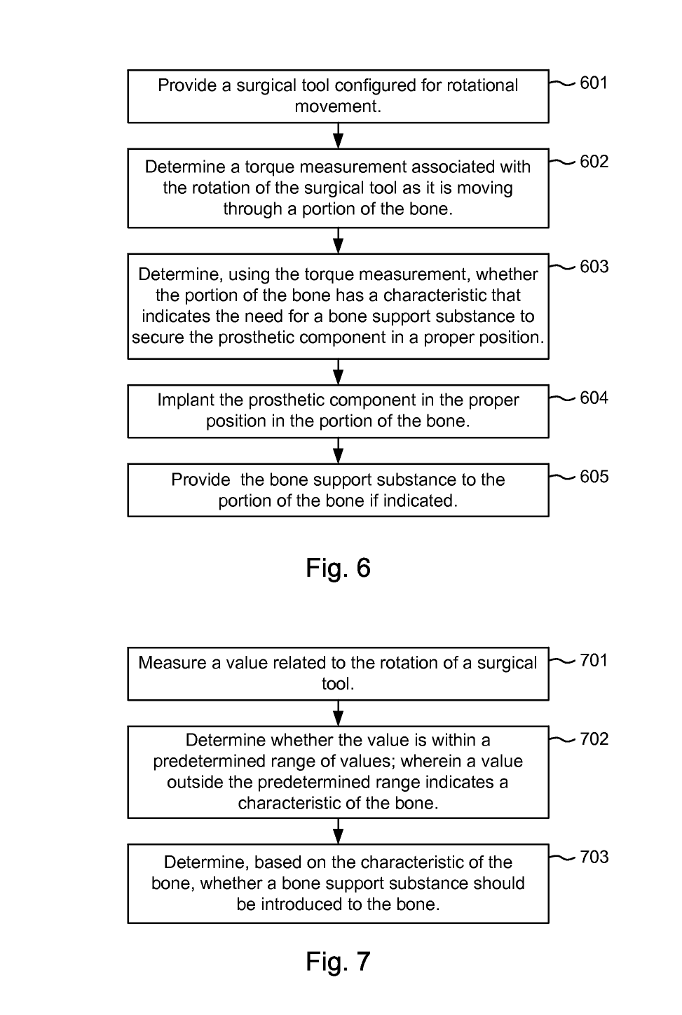

Referring to the flowchart of FIG. 6, described is a method for implanting a prosthetic component in accordance with an exemplary embodiment. In step 601, a surgical tool configured for rotational movement is provided. In an exemplary embodiment, the surgical tool is a tool configured for creating voids in the bone, for example, in preparation for receiving a bone fixation device or in preparation for receiving a prosthetic component. In step 602, a torque measurement associated with the rotation of the surgical tool is determined as it is moving through a portion of the bone. The torque measurement is then used to determine whether the portion of the bone has a characteristic that indicates the need for a bone support substance, such as a bone void filler to secure the prosthetic component in a proper position (step 603). In step 604, the prosthetic component is implanted in the proper position in the portion of the bone. The prosthetic component may be a bone fixation device, such as a screw, nail, tack, pin, or the like. The prosthetic component may also be a joint prosthesis device. In step 605, the bone void filler or bone cement is provided (as necessary or indicated) to the portion of the bone to secure the proper position of the component implanted in the portion of the bone.

Referring to the flowchart of FIG. 7, described is a method for determining an indication for use of a bone support substance , such as a bone void filler in a bone in accordance with an exemplary embodiment. In step 701, a value related to the rotation of a surgical tool is measured. In step 702, it is determined whether the value is within a predetermined range of values, wherein a value outside the predetermined value indicates a characteristic of the bone. For example, the value may be the drilling torque and the predetermined range of values may be torque greater than a minimum torque. The minimum torque may represent the critical value where bone density is low and would not have the strength to retain, for example, a prosthetic component. As such, the torque outside this range of values indicates the low bone density. In step 703, it is determined, based on the characteristic of the bone, whether a bone support substance should be introduced to the bone.

Further modifications and alternative embodiments of various aspects of the invention will be apparent to those skilled in the art in view of this description. Accordingly, this description is to be construed as illustrative only. The construction and arrangements of the bone screws and inserts, as shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, or method steps may be varied or re-sequenced according to alternative embodiments. Other substitutions, modifications, changes and omissions may also be made in the design, operating conditions and arrangement of the various exemplary embodiments without departing from the scope of the present disclosure.

What is claimed is:

1. A method for determining an indication for use of a bone support substance in a bone, comprising:
providing a surgical device configured for providing rotational movement of a surgical tool;
determining a torque measurement associated with the rotation of the surgical tool as it is moving through a portion of the bone; and
using the torque measurement to determine whether the portion of the bone has a characteristic that indicates the need for a bone support substance.

2. The method of claim 1, wherein the bone support substance is a bone void filler.

3. The method of claim 1, wherein the bone support substance is a bone cement.

4. The method of claim 1, wherein the characteristic is a low bone density.

5. The method of claim 4, wherein the torque measurement indicates a bone mass density of 2.5 standard deviations or more below an established healthy bone reference mean.

6. The method of claim 1, wherein the characteristic is a low bone hardness.

7. The method of claim 1, wherein determining a torque measurement comprises determining a motor torque, wherein the motor torque is the torque required to cause rotation of the surgical tool as it is moving through the portion of the bone.

8. The method of claim 7, wherein determining a torque measurement comprises determining the electric current required by the motor to cause rotation of the surgical tool as it is moving through a portion of the bone.

9. The method of claim 1, wherein determining a torque measurement comprises determining, using a torque sensor, a drilling torque, wherein the drilling torque is the force created by the portion of the bone that acts against a motor torque causing the rotation of the surgical tool, as the surgical tool is moving through the portion of the bone.

10. The method of claim 1, wherein the surgical tool is at least one of a burr, a screw, a drill, a reamer, and a tapper.

11. The method of claim 1, further comprising determining the torque measurement at a plurality of portions of the bone as the surgical device moves through the bone, and further comprising determining which portions of the bone have a characteristic that indicates the need for the bone support substance.

12. The method of claim 11, wherein the plurality of portions include a cortical bone portion and a cancellous bone portion.

13. The method of claim 1, further comprising displaying the torque measurement on a display feature of the surgical device.

14. The method of claim 1, further comprising providing an alert to the user that the torque measurement has reached a predetermined torque value.

15. The method of claim 1, wherein the surgical device is a torque wrench.

16. A method for implanting a prosthetic component, comprising:
providing a surgical tool configured for rotational movement;
determining a torque measurement associated with the rotation of the surgical tool as it is moving through a portion of the bone;
using the torque measurement to determine whether the portion of the bone has a characteristic that indicates the need for a bone support substance to secure the prosthetic component in a proper position;
providing the bone support substance to the portion of the bone if so indicated by the torque measurement; and
implanting the prosthetic component in the proper position in the portion of the bone.

17. The method of claim 16, wherein determining that the portion of the bone has a characteristic that indicates the need for a bone support substance comprises determining that the torque measurement is below a predetermined torque value.

18. The method of claim 16, further comprising determining the torque measurement at a plurality of portions of the bone as the surgical device moves about the bone, and further comprising determining which portions of the bone have a characteristic that indicates the need for the bone support substance.

19. A method for determining an indication for use of a bone support substance in a bone, comprising:
measuring a value related to the rotation of a surgical tool;
determining whether the value is within a predetermined range of values, wherein a value outside the predetermined value indicates a characteristic of the bone; and
determining, based on the characteristic of the bone, whether a bone support substance should be introduced to the bone.

20. The method of claim 19, wherein the value is at least one of a torque associated with a rotating portion of the surgical tool, a torque associated with a motor causing the rotation of the surgical tool, an angular velocity of the surgical tool, and a current required to cause the rotation of the surgical tool.
